SEQUENCE LISTING (13 sequences)

SEQ ID NO: 1 - RNA, 19 nt, Artificial
  siRNA derived from survivin
aaggagaucaacauuuuca

SEQ ID NO: 2 - RNA, 19 nt, Artificial
  siRNA derived from survivin
uaggaaaggagaucaacau

SEQ ID NO: 3 - RNA, 19 nt, Artificial
  siRNA derived from survivin
aggaaaggagaucaacauu

SEQ ID NO: 4 - RNA, 19 nt, Artificial
  siRNA derived from survivin
aggaaaggagaucaacauu

SEQ ID NO: 5 - RNA, 19 nt, Artificial
  siRNA derived from survivin
ggaaaggagaucaacauuu

SEQ ID NO: 6 - RNA, 19 nt, Artificial
  siRNA derived from survivin
gaaaggagaucaacauuuu

SEQ ID NO: 7 - RNA, 19 nt, Artificial
  siRNA derived from survivin
aaaggagaucaacauuuuc

SEQ ID NO: 8 - RNA, 19 nt, Artificial
  siRNA derived from survivin
aggagaucaacauuuucaa

SEQ ID NO: 9 - RNA, 19 nt, Artificial
  siRNA derived from survivin
ggagaucaacauuuucaaa

SEQ ID NO: 10 - DNA, 23 bp, Artificial
  survivin specific forwarding primer
ccttcacatctgtcacgttctcc

SEQ ID NO: 11 - DNA, 23 bp, Artificial
  survivin specific reverse primer
atcatcttacgccagacttcagc

SEQ ID NO: 12 - DNA, 20 bp, Artificial
  GAPDH specific forwarding primer
ggtgaaggtcggagtcaacg

SEQ ID NO: 13 - DNA, 25 bp, Artificial
  GAPDH specific reverse primer
accatgtagttgaggtcaatgaagg

(19) United States
(12) Patent Application Publication
(10) Pub. No.: US 2012/0220647 A1
(43) Pub. Date: Aug. 30, 2012

(54) NANO-HYBRID OF TARGETABLE SIRNA-LAYERED INORGANIC HYDROXIDE, MANUFACTURING METHOD THEREOF, AND PHARMACEUTICAL COMPOSITION FOR TREATING TUMOR COMPRISING THE NANO-HYBRID

(76) Inventors: Jin-Ho Choy, Seoul (KR); Dae-Hwan Park, Daejeon (KR); Jaeyong Cho, Seoul (KR)

(21) Appl. No.: 13/496,108

(22) PCT Filed: Sep. 14, 2009
(86) PCT No.: PCT/KR2009/005221

§ 371 Date: May 10, 2012

(51) Int. Cl. A61K 31/7088; A61P 35/00

(52) U.S. Cl. 514/44 A

(57) ABSTRACT

A nanohybrid of the potent gene therapeutic agent siRNA (small interfering RNA) and a target-specific layered inorganic hydroxide, a preparation method thereof, and a pharmaceutical composition for tumor treatment containing the target-specific, siRNA/layered inorganic hydroxide nanohybrid. The nanohybrid increases the in vivo stability of the siRNA, and a target-specific multifunctional ligand, which is bonded to the layered inorganic hydroxide and can bind specifically to a tumor, increases the efficiency of tumor-specific transfer of the siRNA such that the siRNA shows tumor therapeutic activity even at a relatively low dose. Thus, the nanohybrid will be widely useful for target-specific antitumor therapies.

### TECHNICAL FIELD

The present invention relates to a nanohybrid of the potent gene therapeutic agent siRNA (small interfering RNA) and a target-specific layered inorganic hydroxide, a preparation method thereof, and a pharmaceutical composition for tumor treatment containing the target-specific, siRNA/layered inorganic hydroxide nanohybrid.

### BACKGROUND ART

For gene therapy, safe and efficient gene delivery technology has been studied for a long time, and various gene delivery systems and technologies have been developed. Gene delivery technologies developed to date include gene delivery technologies that use viruses such as adenovirus and retrovirus, and gene delivery technologies that use nonviral vectors with liposomes and cationic lipids and polymers. However, methods developed to date, which use viruses themselves as delivery systems for gene therapy, cannot ensure that the gene transferred into the chromosome of a host neither changes the normal function of the host gene, nor activates oncogenes in the host. In addition, even when a small amount of the viral gene continues to be expressed, it can cause autoimmune diseases. Also, when infection with a mutant virus derived from the viral delivery system occurs, protective immunity cannot be efficiently generated. For these reasons, in place of the method that uses virus, a method that uses a gene fused with liposome, a method that uses a cationic lipid or polymer, a method that uses inorganic nanoparticles and the like have been studied in order to overcome the respective disadvantages. These non-viral vectors are significantly less efficient than viral vectors, but have advantages of reduced side effects (high in vivo safety) and low production cost (high cost-effectiveness). At present, in studies on gene delivery systems, approaches about target specific delivery are receiving the greatest attention. When a gene is administered directly in vivo, all the organs and cells in vivo will be attacked by the gene, and thus normal cells and tissues will be damaged. For this reason, the development of technology for selective gene delivery and therapy is important.

Meanwhile, since siRNA was recently found to exhibit an excellent effect on the inhibition of the expression of a specific gene in animal cells, it has received attention as a gene therapeutic agent. Such siRNA has been studied for the last two decades by virtue of its high activity and precise gene selectivity, and is expected to be an alternative therapeutic agent to an antisense oligonuceotide (ODN) which is currently being used as a therapeutic agent. The siRNA is a short double-spiral RNA strand consisting of about 19-23 nucleotides, and targets the mRNA of a gene to be treated, which has a nucleotide sequence complementary thereto, thus inhibiting the expression of the gene. However, siRNA entails a problem in that it is degraded in vivo within a short time due to its low stability and its therapeutic efficiency is deteriorated rapidly. For this reason, expensive siRNA needs to be administered at a high dose. In addition, because siRNA is anionic in nature, it cannot easily permeate the anionic cell membrane, suggesting the intracellular delivery thereof is insufficient (Celia M. &Henry, Chemical and Engineering News December, 22, 32-36, 2003). Further, although siRNA consists of a double strand, the bonds between ribose sugars of RNA are chemically unstable compared to the bonds between deoxyribose sugars of DNA. Thus, siRNA has an in vivo half-life of about 30 minutes and is degraded rapidly in vivo. In recent years, attempts have been made to introduce various functional groups into siRNA so as to protect siRNA from enzymes, thereby improving the stability of siRNA (see Frank Czauderna et al., Nucleic Acids Research 31, 2705-2716, 2003). However, it is considered that technology for ensuring the stability of siRNA and the efficient permeation of siRNA through the cell membrane still remains in the development stage. In addition, in order to obtain the therapeutic effect of siRNA, a method of continuously injecting a high concentration of siRNA in view of the unstability thereof in blood was proposed, but is known to have low efficiency. Furthermore, in order to use siRNA as a gene therapeutic agent in a cost-effective manner, the development of technology for novel non-viral delivery systems that easily delivery siRNA into cells is necessary required. Korean Patent Registration No. 10-0883471 discloses that the use of a hybrid conjugate of siRNA and a hydrophilic polymer covalently linked thereto and the use of a polyelectrolyte complex micelle consisting of the conjugate and a cationic compound can improve the in vivo stability of siRNA to allow the efficient intracellular delivery of therapeutic siRNA, and also enables siRNA to show activity at a relatively low dose.

However, there has been no report on a pharmaceutical composition for tumor treatment which comprises a non-viral layered inorganic hydroxide for improving the stability of siRNA and providing a targeted gene therapeutic agent. Accordingly, the present inventors have made extensive efforts to develop a targeted gene therapeutic agent using siRNA, and as a result, have found that a nanohybrid of siRNA and a layered inorganic hydroxide having a tumor-specific multifunctional ligand bonded thereto can efficiently treat a tumor, thereby completing the present invention.

### DISCLOSURE OF INVENTION

It is an object of the present invention to provide a nanohybrid for improving the efficiency of intracellular delivery of siRNA, which comprises siRNA intercalated in a layered inorganic hydroxide having bonded thereto a target-specific multifunctional ligand capable of binding specifically to a tumor marker, and a method for preparing the same.

Another object of the present invention is to provide a pharmaceutical composition for tumor treatment, containing a target-specific, siRNA/layered inorganic hydroxide nanohybrid together with a pharmaceutically acceptable carrier.

To achieve the above objects, the present invention provides a target-specific, siRNA/layered inorganic hydroxide nanohybrid represented by the following formula 1:

[M(II)₁₋ₓM(III)ₓ(OH)₂]^{X+}[S][T] [Formula 1]

wherein M(II) represents a divalent metal cation, M(III) represents a trivalent metal cation, x is a number ranging from 0.1 to 0.5, S is siRNA, and [T] is a tumor-targeted multifunctional ligand.
The present invention also provides a method for preparing a target-specific, siRNA/layered inorganic hydroxide nanohybrid, the method comprising the steps of: (a) adding an aqueous solution of a base dropwise to an aqueous solution containing a divalent metal salt and a trivalent metal salt to prepare a precipitated layered inorganic hydroxide; (b) mixing an siRNA-containing solution with a dispersion of the layered inorganic hydroxide prepared in step (a), and stirring the mixture, thereby preparing an siRNA/layered inorganic hydroxide nanohybrid; and (c) bonding a tumor marker-specific multifunctional ligand to the nanohybrid, thereby preparing a target-specific, siRNA/layered inorganic hydroxide nanohybrid.

The present invention also provides a pharmaceutical composition for tumor treatment, containing said nanohybrid, and a preparation method thereof.

Other features and embodiments of the present invention will be more apparent from the following detailed descriptions and the appended claims

### BRIEF DESCRIPTION OF THE DRAWINGS

FIG. 1 is a schematic diagram showing a reaction for preparing a target-specific, siRNA/layered inorganic hydroxide nanohybrid.

FIG. 2 is an X-ray diffraction diagram of a target-specific, siRNA/layered inorganic hydroxide nanohybrid, wherein (a): NO₃/layered inorganic hydroxide, (b): siRNA/layered inorganic hydroxide, and (c): target-specific, siRNA/layered inorganic hydroxide nanohybrid.

FIG. 3 is a transmission electron microscope image of a target-specific, siRNA/layered inorganic hydroxide nanohybrid.

FIG. 4 is an electrophoresis image showing the degradation of siRNA with time in the presence of serum protein, taken in order to evaluate the stabilities of siRNA and a target-specific, siRNA/layered inorganic hydroxide nanohybrid in blood, wherein (a): pure siRNA, and (b): target-specific, siRNA/layered inorganic hydroxide nanohybrid.

FIG. 5 is a graphic diagram showing the inhibition of survivin mRNA expression in tumor cells by a target-specific, siRNA/layered inorganic hydroxide nanohybrid, wherein (a): a control; (b): NO₃-layered inorganic hydroxide, (c) siRNA/layered inorganic hydroxide, and (d): target-specific, siRNA/layered inorganic hydroxide nanohybrid in medium containing folic acid; (e): target-specific, siRNA/layered inorganic hydroxide nanohybrid in medium containing no folic acid.

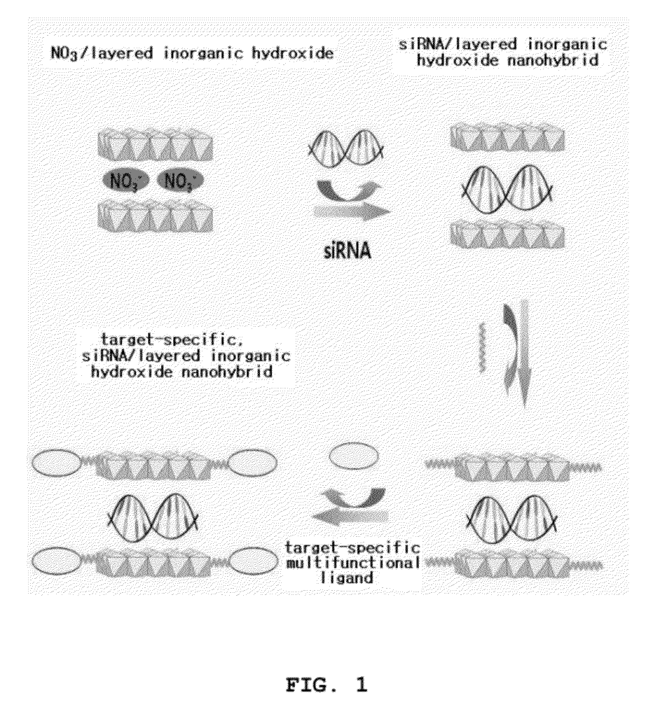

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods which will be described later are those well known and commonly employed in the art.

The present invention is directed to a target-specific, siRNA/layered inorganic hydroxide nanohybrid.

The term “nanohybrid” as used herein means a configuration wherein siRNA is bonded to the layered inorganic hydroxide by intermolecular interaction. Examples of said intermolecular interaction include electrostatic interaction, hydrophobic interaction, hydrogen bonding, covalent bonding (e.g., disulfide bonding), van der Waals bonding, ionic bonding, and the like. In addition, the term “nanohybrid” also means a configuration wherein a target-specific multifunctional ligand is bonded to an siRNA/layered inorganic hydroxide nanohybrid by intermolecular interaction.

In one aspect, the present invention is directed to a target-specific, siRNA/layered inorganic hydroxide nanohybrid represented by the following formula 1:

[M(II)₁₋ₓM(III)ₓ(OH)₂]^{X+}[S][T] [Formula 1]

wherein M(II) represents a divalent metal cation, M(III) represents a trivalent metal cation, x is a number ranging from 0.1 to 0.5, S is siRNA, and [T] is a tumor-targeted multifunctional ligand.

In the present invention, in the case of siRNA having a long nucleotide length (for example, a molecular weight of about 30,000), the double-strand siRNA oligomer can stably bind to the RNA-induced silencing complex (RISC), an enzyme complex that is involved in the gene inhibitory activity of the siRNA, even when it is bonded to a target-specific layered metal hydroxide. However, in the case of siRNA consisting of 19 nucleotides (corresponding to a molecular weight of about 10,000), when it binds with an intracellular enzyme complex that assists in the gene inhibitory activity, the structural stability thereof can be reduced by a target-specific multifunctional ligand. For this reason, it is advantageous to introduce a linkage that can be cleaved in vivo or in cells. Thus, the siRNA oligomer has a molecular weight ranging from 10,000 to 30,000. Within this range, siRNA comprises 19 to 30 nucleotides, preferably 19 to 23 nucleotides. Preferred examples of the siRNA include, but are not limited to, siRNAs derived from c-myc, c-myb, c-fos, c-jun, c-raf, c-src, bcl-2, vascular endothelial growth factor (VEGF), VEGF-B, VEGF-C, VEGF-D, PIGF, or survivin.

Also, the siRNA is preferably introduced simultaneously with a disulfide bond which is degraded in cells by glutathione that is present in an excessive amount in the cytoplasm, an acid cleavable bond which can be effectively cleaved in an acidic environment after introduction into cells, an ester bond or an anhydride bond which can be effectively cleaved in cells after introduction into cells, or an enzyme-cleavable bond that can be cleaved by enzymes, which exist around cells, immediately before introduction into cells.

Generally, cancer develops from the decreased rate of apoptosis which is an active and voluntary cell death, and from the altered cell cycles. Thus, a method of recovering the apoptotic process or suppressing the cell cycles is receiving attention as a new tumor therapeutic method. It is known that inhibitors of apoptosis (IAPs) are expressed in tumors in which apoptosis is suppressed. It is known that the IAPs show their activity by directly inhibiting the activity of apoptosis-inducing protease (caspase) or regulating the activity of the related transcription factor NF-kB. A recent study revealed that surviving protein, one of IAPs, is associated with tumors. It is known that survivin is commonly expressed in most of new tumors or transformed cells and is also expressed in tumors in which continuous mutation occurs. Thus, survivin is expected to be an important target in anticancer therapy (see: Ambrosini G, et al., Nat. Med., 3(8):917-921, 1997).

Attention has been paid to a method in which an siRNA, which can bind to an mRNA transcribed from a survivin-encoding gene to inactivate the mRNA, is introduced directly into tumor cells to inhibit the expression of survivin in the tumor cells or suppress the activity of survivin in the tumor cells, thereby effectively treating the cancer cells (Korean Patent Registration No. 10-0848665). The double-strand siRNA of the present invention can bind to an mRNA transcribed from a survivin-encoding gene and inhibits the expression of survivin in cells.

A preferred example of the siRNA of the present invention may be an siRNA which can bind complementarily to a survivin-encoding mRNA and can inhibit the expression of survivin that is commonly expressed in almost all tumor cells.

Specifically, the siRNAs which can bind complementarily to the survivin-encoding mRNA may have the nucleotide sequences shown in Table 1 below.

TABLE 1

| Nucleotide sequences | SEQ ID NOs |
|---|---|
| 5′-AAGGAGAUCAACAUUUUCA-3′ | SEQ ID NO: 1 |
| 5′-UAGGAAAGGAGAUCAACAU-3′ | SEQ ID NO: 2 |
| 5′-AGGAAAGGAGAUCAACAUU-3′ | SEQ ID NO: 3 |
| 5′-AGGAAAGGAGAUCAACAUU-3′ | SEQ ID NO: 4 |
| 5′-GGAAAGGAGAUCAACAUUU-3′ | SEQ ID NO: 5 |
| 5′-GAAAGGAGAUCAACAUUUU-3′ | SEQ ID NO: 6 |
| 5′-AAAGGAGAUCAACAUUUUC-3′ | SEQ ID NO: 7 |
| 5′-AGGAGAUCAACAUUUUCAA-3′ | SEQ ID NO: 8 |
| 5′-GGAGAUCAACAUUUUCAAA-3′ | SEQ ID NO: 9 |

Also, the sense or antisense end of the siRNA may be substituted with other functional groups. For example, the 3′ hydroxyl group of the siRNA may be substituted with an amine group, a sulfhydryl group or a phosphate group. The siRNA according to the present invention may further comprise a tumor cell-selective ligand. Preferred examples of the ligand include cell specific antibody, cell selective peptide, cell growth factor, folic acid, galactose, mannose, RGD, and transferrin.

These ligands may be introduced into the terminal end of the siRNA by a bond, such as a disulfide bond, an amide bond or an ester bond.

In the present invention, the layered inorganic hydroxide has a layered crystal structure and anion exchange capacity. This is because the hydroxide layer of the layered inorganic hydroxide bears a positive charge, and thus an anion is present between the layers in order to compensate for the positive charge, and the interlayer anion can be substituted with other anionic chemical species. The layered inorganic hydroxide may be represented by the following formula 2:

[M(II)₁₋ₓM(III)ₓ(OH)₂]^{X+}[Aⁿ⁻]_{X/n}.yH₂O [Formula 2]

wherein M(II) represents a divalent metal cation, M(III) represents a trivalent metal cation, A is an anionic chemical species, n is the charge number of the anion, x is a number ranging from 0.1 to 0.5, and y is a positive number greater than 0.

In the present invention, the divalent metal cation is selected from the group consisting of Mg²⁺, Ca²⁺, Co²⁺, Cu²⁺, Ni²⁺ and Zn²⁺, the trivalent metal cation is selected from the group consisting of Al³⁺, Cr³⁺, Fe³⁺, Ga³⁺, In³⁺, V³⁺ and Ti³⁺, and the anion is selected from the group consisting of CO₃²⁻, NO³⁻, Cl⁻, OH⁻, O²⁻ and SO₄²⁻. The ratio between the divalent metal cation and the trivalent metal cation may be controlled to 2:1, 3:1 and 4:1, thereby forming a layered inorganic hydroxide having controlled layer charge. The divalent metal cation, the trivalent metal cation and the anion are not limited to those as listed above, and examples thereof may include all ions for layered inorganic hydroxides known in the art.

As used herein, the term “tumor-targeted multifunctional ligand” means a tumor-specific binding ingredient which is additionally bonded to the siRNA/layered inorganic layer nanohybrid so as to impart target specificity. Examples of this tumor-specific binding ingredient include, but are not limited to, antigen, antibody, RNA, DNA, hapten, avidin, streptavidin, neutravidin, protein A, protein G, lectin, selectin, a radioisotope-labeled biomaterial, and a biomaterial that can bind specifically to a tumor marker.

Herein, the term “target-specific multifunctional ligand” means a material comprising (i) an attachment region, (ii) a crosslinking region, and (iii) an active ingredient region. Hereinafter, the multifunctional ligand will be described in detail.

As used herein, the term “attachment region” means either a spacer or a portion, preferably an end, of a target-specific multifunctional ligand, which comprises a functional group and can be attached to the layered inorganic hydroxide so as to modify surface modification of the hydroxide. Thus, the attachment region preferably comprises a functional group having high affinity for the surface of the layered inorganic hydroxide and may be suitably selected depending on the material of the layered inorganic hydroxide. The attachment region may comprise, for example, aminosilane, epoxysilane, vinylsilane, —COOH, —NH₂, —SH, —CONH₂, —PO₃H, —PO₄H, —SO₃H, —SO₄H or —OH.

As used herein, the term “crosslinking region” means the ‘end of the attachment region’ and the ‘end of the target-specific multifunctional ligand’, which comprise a functional group that can crosslink with another functional group at a portion of the multifunctional ligand adjacent to the surface-modified layered multifunctional ligand. The term “crosslinking” means the bonding of the multifunctional ligand to the end of the attachment region, attached to the surface-modified layered inorganic hydroxide, by intermolecular interaction. This intermolecular interaction is various, including hydrophobic interaction, hydrogen bonding, covalent bonding (e.g., disulfide bonding), van der Waals bonding, ionic bonding, and the like, and thus a functional group which can be used for this crosslinking can be suitably selected depending on the desired type of intermolecular interaction. The crosslinking region may comprise a functional group selected from among, for example, —SH, —NH₂, —COOH, —OH, —NR₄⁺X⁻, epoxy, -ethylene, -acetylene, -sulfonate, -nitrate, and phosphonate. The functional group of the crosslinking region can vary depending on the end of the attachment region and the kind and chemical formula of the active ingredient region.

Said intermolecular linkage may be any of a non-cleavable linkage or a cleavable linkage. Herein, examples of the non-cleavable linkage include, but are not limited to, an amide bond and a phosphate bond, and examples of the cleavable linkage include, but are not limited to, a disulfide bond, an acid-cleavable linkage, an ester bond, an anhydride bond, a biodegradable bond, and an enzyme-cleavable linkage.

The term “active ingredient region” means a tumor-specific binding ingredient or a portion of the target-specific multifunctional ligand (preferably a portion located opposite to the attachment region), which comprises a functional group capable of crosslinking with the attachment region.

Herein, examples of the tumor-specific binding ingredient include, but are not limited to, antigen, antibody, RNA, DNA, hapten, avidin, streptavidin, neutravidin, protein A, protein G, lectin, selectin, a radioisotope-labeled component, and a material that can bind specifically with a tumor marker. Preferred examples of the tumor-specific binding ingredient include cell-specific antibody, cell-selective peptide, cell growth factor, folic acid, galactose, mannose, RGD, and transferrin. The active ingredient may preferably be a ligand or an antibody, which can bind specifically to a tumor. Examples of the ligand include cell-specific antibody, cell-selective peptide, cell growth factor, folic acid, galactose, mannose, RGD, and transferrin.

The present invention also provides a nanohybrid wherein a therapeutic substance that can bind specifically to a tumor is bonded to the target-specific, siRNA/layered inorganic hydride nanohybrid. As used herein, the term “target-specific, siRNA/layered inorganic hydroxide nanohybrid” means a nanohybrid wherein the siRNA/layered inorganic hydroxide nanohybrid is surrounded by the target-specific multifunctional ligand comprising the attachment region, the crosslinking region and the active ingredient region, in which a substance that can bind specifically to a tumor marker is bonded to the active ingredient region.

In a referred embodiment of the present invention, as the target-specific multifunctional ligand of the target-specific nanohybrid, a folic acid was used, which has a carboxyl end and responds selectively to the folate receptor that is overexpressed in tumor cells. Specifically, the attachment region is the silane moiety of aminosilane, and the crosslinking region is a peptide region formed by reaction of the amine moiety of aminosilane with the carboxyl end of folic acid, and the active ingredient region is a region which responds to the folate receptor.

Folic acid (FA) is a nutrient playing an important role in the folate cycle, a mechanism that produces a gene in a cell. Particularly, it is known to play an important role in cell differentiation. Generally, cancer cells require a large amount of folic acid (or folate) for rapid cell differentiation, and thus tend to overexpress the folate receptor in the cell membrane in order to take a large amount of folic acid. Particularly, the folate receptor is more expressed in some breast cells (such as KB cells) than in normal cells, and thus folate can function as a kind of ligand that recognizes these cancer cells. Examples of ligands that recognize cancer cells include, in addition to chemical substances such as folate, antibodies, aptamers and the like. However, folate is highly advantageous as a ligand, because it cause no immune side effects and is relatively inexpensive. Thus, in recent several studies, there were efforts to use folate as a ligand to increase the affinity of drug delivery systems for cancer cells. Typical examples of such studies include a study carried out to attach a ligand to the surface of a drug delivery system such as liposome (Gabizon, A. et al., Adv. Drug Delivery Rev. (2004) 56:1177-1192), a study carried out to attach a ligand to the end of a polymeric drug delivery system such as PEG-DSPE (polyethyelenglycol-disterarolyl phosphatidylethanolamine) in order to increase the efficiency of transfection of DNA (Hattori, Y. et al., J. Contorlled Rel., (2004) 97:173-183), and a study carried out to attach folate to a hydrogen-type drug delivery system such as pNIPAM (poly(N-isopropylacrylamide)) in order to target cells (Nayak, S. et al., J. Am. Chem. Soc., (2004) 126:10258-10259).

The nanohybrid of the present invention can be used as a gene therapeutic agent for treating a variety of tumor-related diseases, for example, stomach cancer, lung cancer, breast cancer, ovarian cancer, liver cancer, bronchogenic carcinoma, nasopharyngeal cancer, laryngeal cancer, pancreatic cancer, bladder cancer, colon cancer, and cervical cancer.

Cells having the above-described tumor diseases express and/or secrete a specific substance which is seldom or never produced in normal cells. This substance is called “tumor marker”. A nanohybrid prepared by bonding a substance, which can bind specifically to this tumor marker, to the siRNA/layered inorganic hydroxide, may be useful for treatment of tumors. As is known in the art, there are a variety of tumor markers and substances that can bind specifically to these tumor markers. The ligand that is used in the present invention may preferably be cell-specific antibody, cell-selective peptide, cell growth factor, folic acid, galactose, mannose, RGD, transferrin or the like.

In the present invention, the tumor markers can be classified according to mechanism into ligands, antigens, receptors, and nucleic acids that encode them.

When the tumor marker is a ligand, a substance that can bind specifically to the ligand may be introduced as a target-specific multifunctional ligand ingredient into the nanohybrid of the present invention and is preferably a receptor or an antibody, which can bind specifically to the ligand. Examples of a combination of a ligand that may be used in the present invention and a receptor that can bind specifically thereto include, but are not limited to, synaptotagmin C2 and phosphatidylserine, annexin V and phosphatidylserine, integrin and its receptor, VEGF (Vascular Endothelial Growth Factor) and its receptor, angiopoietin and Tie2 receptor, somatostatin and its receptor, and nasointestinal peptide and its receptor.

When the tumor marker is an antigen, a substance that can bind specifically to the antigen may be introduced as a target-specific active ingredient into the nanohybrid of the present invention and is preferably an antibody that can bind specifically to the antigen. Examples of a combination of an antigen that may be used in the present invention and an antibody that binds specifically thereto include, but are not limited to, carcinoembryonic antigen (colorectal cancer marker antigen) and Herceptin (Genentech, USA), HER2/neu antigen (breast cancer marker antigen) and Herceptin, and prostate-specific membrane antigen (prostate cancer marker antigen) and Rituxan (IDCE/Genentech, USA).

Typical examples of a tumor marker which is a receptor include folic acid receptor which is expressed in ovarian cancer cells. A substance that can bind specifically to the receptor (folic acid for folic acid receptor) may be introduced as a target-specific multifunctional ligand into the nanohybrid of the present invention and is preferably a ligand or an antibody, which can bind specifically to the receptor. More preferably, it is an antibody. As described above, an antibody is particularly preferred active ingredient in the present invention. This is because an antibody has the ability to selectively and stably bind only to a specific subject, and —NH₂ of lysine, —SH of cysteine, and —COOH of aspartate and glutamate, which are present in the Fc region of the antibody, may be useful for binding to a functional group at the active ingredient-binding region of the nanohybrid. Such antibodies may be commercially available or may be prepared by any method known in the art. Generally, a mammal (e.g., mice, rats, goats, rabbits, horses or sheep) is immunized with a suitable amount of an antigen once more. When the antibody titer has reached a suitable level after a given period of time, an antibody is collected from the serum of the mammal. The collected antibody may be, if desired, purified using a known process, and may be stored in a freezing buffer solution until use. The details of such a method are well known in the art.

Meanwhile, the term “nucleic acids” includes RNA and DNA encoding the aforementioned ligands, antigens, receptors, or portions thereof. Nucleic acids, as known in the art, form base pairs between complementary sequences. Thus, a nucleic acid having a specific nucleotide sequence may be detected using another nucleic acid having a nucleotide sequence complementary to the nucleotide sequence. Nucleic acids having nucleotide sequences complementary to nucleic acids encoding the aforementioned enzymes, ligands, antigens and receptors may be used as the target-specific active ingredient of the nanohybrid according to the present invention.

In addition, nucleic acids may be useful for binding to a functional group of the active ingredient-binding region because they have functional groups, such as —NH₂, —SH and —COOH, at their 5′-end and 3′-end. Such nucleic acids may be synthesized by any standard method known in the art, for example, using an automated DNA synthesizer (such as commercially available from Biosearch, Applied Biosystems, etc.). For example, phosphorothioate oligonucleotides may be synthesized using the method described in tie literature (Stein et al., Nucl. Acids Res. 1988, vol. 16, p. 3209). Methylphosphonate oligonucleotides may be prepared using controlled glass polymer supports (Sarin et al. Proc. Natl. Acad. Sci. U.S.A. 1988, vol. 85, p. 7448).

In the present invention, the particle size of the nanohybrid is preferably 10-350 nm, and more preferably 50-200 nm. In this case, the target-specific nanohybrid responds selectively to the receptor of the multifunctional ligand, that is, a tumor marker, and then is effectively incorporated in cells. In addition, when it is administered in vivo, it does not block capillary blood vessels and has no physical influence on cells. If the target-specific nanohybrid has a particle size of less than 50 nm, it can be introduced into cells in large amount such that it can have a physical influence on cells.

In the present invention, the content of siRNA in the target-specific, siRNA-layered inorganic hydroxide nanohybrid is preferably 1-50 wt %.

In another aspect, the present invention is directed to a method for preparing a target-specific, siRNA/layered inorganic hydroxide nanohybrid, the method comprising the steps of: (a) adding an aqueous solution of a base dropwise to an aqueous solution containing a divalent metal salt and a trivalent metal salt to prepare a precipitated layered inorganic hydroxide; (b) mixing an siRNA-containing solution with a dispersion of the layered inorganic hydroxide prepared in step (a), and stirring the mixture, thereby preparing an siRNA/layered inorganic hydroxide nanohybrid; and (c) bonding a tumor marker-specific multifunctional ligand to the nanohybrid, thereby preparing a target-specific, siRNA/layered inorganic hydroxide nanohybrid.

In addition, the present invention is directed to a method for preparing a pharmaceutical composition for tumor treatment containing said nanohybrid, the method comprising step of (d) formulating the nanohybrid, obtained in step (c) of the above method, with one or morepharmaceutically acceptable carriers.

In the present invention, the layered inorganic hydroxide in step (a) is represented by the following formula 2 and may be easily prepared by a co-precipitation method:

[M(III)₁₋ₓM(III)ₓ(OH)ₓ(OH)₂]^{X+}[Aⁿ⁻]_{X/n}.yH₂O [Formula 2]

wherein M(II) represents a divalent metal cation, M(III) represents a trivalent metal cation, A is an anionic chemical species, n is the charge number of the anion, x is a number range from 0.1 to 0.5, and y is a positive number greater than 0.

In formula 2 as described above, M(III) is a trivalent metal cation and may be present or absent. If the M(II) ion and the M(III) ion coexist as shown in formula 2 above, an excess of the M(III) ion can interfere with production of the layered structure. For this reason, the M(III) ion is preferably present in an amount of 50 mol % or less based on the total amount of the metal ions.

The ratio between the divalent metal cation and the trivalent metal cation may be controlled to 2:1, 3:1 and 4:1, thereby forming a layered inorganic hydroxide having controlled layer charge.

The divalent metal salt that is used in the method of the present invention may be a salt compound which comprises Mg²⁺, Ca²⁺, Co²⁺, Cu²⁺, Ni²⁺ or Zn²⁺ as a cation and NO³⁻, Cl⁻, OH⁻, O²⁻, SO₄²⁻, CO₃²⁻ or succinate as an anion, but is not limited thereto. The trivalent metal salt that is used in the present invention may be a salt compound which comprises Al³⁺, Cr³⁺, Fe³⁺, Ga³⁺, In³⁺, V³⁺ or Ti³⁺ as a cation and NO³, Cl⁻, OH⁻, O²⁻, SO₄²⁻, CO₃²⁻ or succinate as an anion, but is not limited thereto. For metal salts of Mg, Mg(NO₃)₂, MgCl₂, MgSO₄, or hydrates thereof may be used, and for metal salts of Al, Al(OH)₃, Al(NO₃)₃, Al₂(SO₄)₃, or hydrates thereof may be used.

The divalent metal salt, the trivalent metal salt, and the anion are not limited to the above examples, and may include all those that correspond to examples known as the layered inorganic hydroxide in the art.

In the coprecipitation reaction, precipitation can be induced by adding a base. Examples of a suitable base that may be used in the present invention include sodium hydroxide, potassium hydroxide, magnesium hydroxide, calcium hydroxide, and ammonia. The pH of the reaction solution is 5-12, and preferably 6-10, and the reaction temperature is 0° C. to 100° C., and preferably 15° C. to 30° C. The reaction time is preferably 10 minutes or more. In addition, nitrogen or inert gas is preferably continuously supplied during the reaction.

The layered inorganic hydroxide can show various particle sizes and shapes depending on various factors of the preparation process, including i) the temperature of the reaction solution, ii) the concentration of the reaction solution, iii) the mixing ratio between metal cations, iv) the temperature of washing water, and v) drying temperature.

In the present invention, the preparation of the siRNA/layered inorganic hydroxide nanohybrid in step (b) may be performed by any one of the following methods: a co-precipitation method in which the siRNA-containing solution is co-precipitated with the prepared layered inorganic hydroxide, thereby forming the nanohybrid; an ion-exchange method in which the formed layered inorganic hydroxide is mixed with the siRNA-containing solution and stirred, and the stirred mixture is introduced between the layers of the layered inorganic hydroxide by ion exchange, thereby forming the nanohybrid; a calcination-reconstruction method in which the prepared layered inorganic hydroxide is calcinned, and then reconstructed by adding the siRNA-containing solution thereto, thereby forming the nanohybrid; and an exfoliation-reassembling method in which the prepared layered inorganic hydroxide is exfoliated into sheets, and then reassembled by adding the siRNA-containing solution thereto, thereby forming the nanohybrid.

The method of preparing the inventive nanohybrid by the co-precipitation method comprises the steps of: preparing a solution of siRNA and divalent/trivalent metal salts; and adding a base to the solution to adjust the pH of the solution to 6-10, thus obtaining a precipitate.

The method of preparing the inventive nanohybrid by the ion-exchange method comprises the steps of: preparing a solution of divalent/trivalent metal salts; adding a base to the solution of divalent/trivalent metal salts to adjust the pH of the solution to 6-10, thereby forming a layered inorganic hydroxide precipitate; and adding an siRNA-containing solution to the formed precipitate to introduce the siRNA between the layers of the precipitate by ion exchange with an anion present between the precipitate.

The method of preparing the inventive nanohybrid by the calcination-reconstruction method comprises the steps of: preparing a solution of divalent/trivalent metal salts; adding a base to the solution of divalent/trivalent metal salts to adjust the pH of the solution to 6-10, thereby forming a layered inorganic hydroxide precipitate; calcining the precipitate at a temperature between 250° C. and 500° C. for 1 hour or more, preferably at a temperature of about 400° C. for about 4 hours, thereby removing an anion between the layers of the precipitate; and adding the calcined precipitate to an siRNA-containing solution and stirring the mixture, thereby reconstructing the precipitate.

The method of preparing the inventive nanohybrid by the exfoliation-reassembling method comprises the steps of: preparing a solution of divalent/trivalent metal salts; adding a base to the solution of divalent/trivalent metal salts to adjust the pH of the solution to 6-10, thereby forming a layered inorganic hydroxide precipitate; either substituting the interlayer anion of the formed precipitate with a long-carbon-chain anion or exfoliating the layered inorganic hydroxide into single-layer sheets using a specific solvent, preferably by dispersing the layered inorganic hydroxide in a formamide solution to a concentration of 0.05 wt % and stirring the dispersion for 2 days, thereby obtaining a colloidal solution; and adding an siRNA-containing solution to the colloidal solution and stirring the mixture, thereby reassembling the precipitate.

The solvent that is used to prepare the solution of divalent/trivalent metal salts, the base solution or the divalent/trivalent metal salt solution in the above preparation methods is not specifically limited, so long as it can dissolve all the siRNA and the metal salts without participating in the reaction. For example, the solvent may be distilled water, ethanol, or a mixed solvent of distilled water and ethanol.

In the above preparation methods, the reaction between the layered inorganic hydroxide and the siRNA is not specifically limited and can generally be carried out at room temperature, preferably a temperature below the denaturation temperature of the siRNA, for about 10 minutes to 7 days. The ratio between the reactants required for the reaction is not specifically limited, and the siRNA and the layered inorganic hydroxide may be used at a molar ratio of 10:90 to 90:10 in the reaction, thereby controlling the rate of introduction of siRNA into the layered inorganic hydroxide.

In addition, after siRNA is transferred into tumor cells, metal cations dissociated from the layered inorganic hydroxide can interact with the phosphate group of the double-strand siRNA to form an insoluble material, which can adversely affect the activity of the siRNA (Duguid J et al., Biophys. J. 65:1916-1928, 1993). When EDTA (ethylene diamine tetraacetic acid) that forms strong chelate bonds with divalent/trivalent metal cations is added together with siRNA in step (b) of preparing the siRNA/layered inorganic hydroxide nanohybrid and the resulting nanohybrid is used for treatment of a tumor and enters a cell, the EDTA can reduce the interaction of the siRNA with metal cations (dissociated from the layered inorganic hydroxide) under weakly acidic conditions, thereby contributing to increasing the activity of the siRNA.

In still another aspect, the present invention is also directed to a pharmaceutical composition for tumor treatment, containing said nanohybrid as an active ingredient, and a preparation method thereof.

The pharmaceutical composition according to the present invention may comprise a pharmaceutically acceptable carrier or vehicle which is conventionally used in the art.

Examples of a tumor that can be treated with the composition of the present invention include, but are not limited to, breast cancer, lung cancer, oral cancer, pancreatic cancer, colon cancer, prostate cancer, ovarian cancer, and the like. Specifically, the tumor may be oral cancer or lung cancer as described in examples below.

Examples of a pharmaceutically acceptable carrier that may be used in the pharmaceutical composition for tumor treatment according to the present invention include, but are not limited to, ion exchange resin, alumina, aluminum stearate, lecithin, serum proteins (e.g., human serum albumin), buffering agents (e.g., sodium phosphate, glycine, sorbic acid, potassium sorbate, partial glyceride mixtures of vegetable saturated fatty acids), water, salts or electrolytes (e.g., protamine sulfate, disodium hydrophosphate, potassium hydrophosphate, sodium chloride, and zinc salts), colloidal silica, magnesium trisilicate, polyvinylpyrrolidone, cellulose-based substrates, polyethylene glycol, sodium carboxymethylcellulose, polyarylate, waxes, polyethylene glycol, and wool fat. In addition to the above components, the pharmaceutical composition for tumor treatment according to the present invention may further comprise lubricants, wetting agents, emulsifiers, suspending agents, preservatives, and the like.

For clinical treatments, the pharmaceutical composition according to the present invention may be formulated into a suitable form using any conventional technique. For example, for oral administration, the composition of the present invention may be mixed with an inert diluent or edible carrier, or filled in a hard or soft gelatin capsule, or compressed into a tablet. For oral administration, an active compound may be mixed with an excipient to form an oral tablet, a buccal tablet, a troche, a capsule, an elixir, a suspension, a syrup, a wafer, or the like. In addition, various formulations for injectable or parenteral administration may be produced using techniques publicly or commonly known in the art.

For administration, the pharmaceutical composition for tumor treatment containing the nanohybrid may comprise, in addition to the above-described active ingredient, one or more pharmaceutically acceptable carriers. The pharmaceutically acceptable carrier must be compatible with the active ingredient of the present invention and may be one or a mixture of two or more selected from among saline, sterile water, Ringer's solution, buffered saline, dextrose solution, malto-dextrin solution, glycerol, and ethanol. If necessary, the composition of the present invention may comprise other conventional additives, such as antioxidants, buffers and bacteriostatic agents. In addition, the composition of the present invention may additionally comprise diluents, dispersants, surfactants, binders and lubricants to provide injectable formulations such as aqueous solutions, suspensions and emulsions. Furthermore, the pharmaceutical composition is preferably formulated according to a disease or component using a method disclosed in Remington's Pharmaceutical Science, Mack Publishing Company, Easton Pa., which is a suitable method in the corresponding field. Moreover, depending on the kind of ingredient or disease, the formulation may be conducted using methods known in the art or disclosed in Remington's Pharmaceutical Science (latest version), Mack Publishing Company, Easton Pa.).

The pharmaceutical composition for tumor treatment containing the nanohybrid according to the present invention may be administered through routes which are conventionally used in the medical field. Preferably, the composition may be administered parenterally, for example, intravenously, intramuscularly, intra-arterially, intramedularry, intrathecally, intraventricularly, transdermally, subcutaneously, intraperitoneally, enterally, sublingually, or topically.

In one embodiment, the pharmaceutical composition for tumor treatment containing the nanohybrid according to the present invention may be formulated into a water-soluble aqueous solution for parenteral administration. Examples of the water-soluble solution include a buffer solution such as Hank's solution, Ringer's solution, or physically buffered saline. Water-soluble injection suspension may contain a substance that can increase the viscosity of the suspension, such as sodium carboxyl methylcellulose, sorbitol, or dextran.

In the present invention, the formulation may be selected from the group consisting of tablets, capsules, liquids, injectable solutions, ointments, and syrups. If the formulation is an injectable solution, it may be in the form of a liquid, a suspension or an emulsion.

The pharmaceutical composition for tumor treatment containing the nanohybrid according to the present invention may be in the form of a sterilized formulation for injection, such as an aqueous or oily suspension. Such a suspension may be prepared using a suitable dispersing agent or wetting agent (for example, Tween 80) and suspending agent according to any technique known in the art. The sterilized formulation for injection may be a sterile injection solution or suspension (for example, a solution in 1,3-butanediol) in a nontoxic and parentally acceptable diluent or solvent. Useable vehicles and solvents include mannitol, water, Ringer's solution, and isotonic sodium chloride solution. Sterile non-volatile oil is generally used as a solvent or a suspending medium. For this purpose, any non-irritating non-volatile oil such as synthetic mono- or di-glyceride may be used.

In addition to a final formulation for injection or infusion, the composition of the present invention may be a dosage form which is present as a lyophilized material or sterilized powder, which may be mixed with, for example, water, immediately before administration, to prepare a final preparation for injection or infusion.

The dosage form of the pharmaceutical composition for tumor treatment containing the nanohybrid according to the present invention can be determined by a person of ordinary skill in the art based on the patient's symptoms and the severity of the disease. In addition, the composition of the present invention can be formulated into various forms, including powders, tablets, capsules, liquids, injectable solutions, and syrups, and can be formulated in the form of a unit-dosage or multi-dosage container, for example, a sealed ampule or bottle.

The dose of the pharmaceutical composition for tumor treatment containing the nanohybrid according to the present invention can vary depending on the weight, age, sex and health condition of a patient (a subject to be treated), the diet, the duration of administration, the mode of administration, the rate of excretion, and the severity of the disease and can be easily determined by a person of ordinary skill in the art.

In the present invention, the dose of the siRNA/layered inorganic hydroxide nanohybrid that can bind complementarily to a gene encoding survivin may be 0.05 to 0.1 μg of siRNA per kg weight depending on the patient's age, sex and symptoms, the method of administration, or preventive purposes. The dose of the composition for a patient showing special symptoms can be determined by a person of ordinary skill in the art depending on the patient's weight, age, sex and health condition, the diet, the duration of administration, the mode of administration, and the like.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Preparation of Target-Specific, siRNA/Layered Inorganic Hydroxide Nanohybrid

1-1: Preparation of NO₃/Layered Inorganic Hydroxide

Mg(NO₃)₂.H₂O (0.2 M) and Al(NO₃)₃.H₂O (0.1 M) were dissolved in carbonate ion (CO₃²⁻)-free distilled water and adjusted to a pH of 9-10 with an NaOH aqueous solution (1 M), thereby obtaining a layered inorganic hydroxide crystal formed by precipitation. The layered inorganic hydroxide crystal was stirred at 100° C. for 16 hours and washed to remove unreacted salts. Then, the precipitate was freeze-dried, thereby obtaining an NO₃/layered inorganic hydroxide.

1-2: Preparation of siRNA/Layered Inorganic Hydroxide Nanohybrid

The layered inorganic hydroxide obtained in Example 1-1 was dispersed in distilled water, and a solution of siRNA (SEQ ID NO: 1; Bioneer, Korea), which can bind complementarily to a survivin-encoding gene, in distilled water, was added to the dispersion (siRNA: layered inorganic hydroxide=3:1 w/w). The mixture was stirred at 37° C. for 2 days and then washed to remove unreacted siRNA, thereby obtaining a nanohybrid comprising siRNA intercalated between the layers of the layered inorganic hydroxide. The preparation of the nanohybrid was carried out in a nitrogen atmosphere in order to prevent carbonate ions (CO₃²⁻) being produced by the carbon dioxide of the air.

1-3: Preparation of Target-Specific, siRNA/Layered Inorganic Hydroxide Nanohybrid

In order to prepare a target-specific, siRNA/layered inorganic nanohybrid having bonded thereto a multifunctional folic acid ligand as an active ingredient, aminosilane was attached to the siRNA/layered inorganic hydroxide prepared in Example 1-2. Specifically, the siRNA-layered inorganic hydroxide was dispersed in ethanol and dried to evaporate the surface water. Then, the hydroxide was added to a solution of aminopropylsilane in toluene and stirred at 60° C. for 6 hours, followed by washing, thereby obtaining an siRNA/layered inorganic hydroxide nanohybrid comprising aminosilane attached to the attachment region. The following solutions were prepared: an aqueous solution of the siRNA/layered inorganic hydroxide nanohybrid having aminosilane bonded thereto; an aqueous solution of each of 1-ethyl-3-(3-dimethylaminopropyl)-carbodiimide (EDC), N-hydroxysuccinimide (NHS) and triethylamine (ET₃N), which are reaction catalysts; and an aqueous solution of folic acid (folate) in dimethylsulfoxide (DMSO). To the aqueous solution of the siRNA/layered inorganic hydroxide nanohybrid, the aqueous solution of 1-ethyl-3-(3-dimethylaminopropyl)-carbodiimide and the aqueous solution of N-hydroxysuccinimide were added and the solution of folic acid was added thereto. Then, the mixed solution was adjusted to a pH of 9 using the aqueous solution of triethylamine. Then, the reaction solution was stirred at 38° C. for 5 hours, washed with dimethylsulfoxide and distilled water and then freeze-dried, thereby obtaining a target-specific, siRNA/layered inorganic hydroxide nanohybrid having bonded thereto a multifunctional folic acid ligand.

The above surface reaction for preparing the target-specific, siRNA/layered inorganic hydroxide nanohybrid is as follows. First, in the attachment region, the M (metal)-OH bond of the layered inorganic hydroxide is surface-modified into M-O—Si-amine. Then, in the crosslinking region, the amine group of M-O—Si-amine reacts with the carboxylic acid to form M-O—Si-peptide-folic acid. The active ingredient region is the end of M-O—Si-peptide-folic acid that responds to the folate receptor.

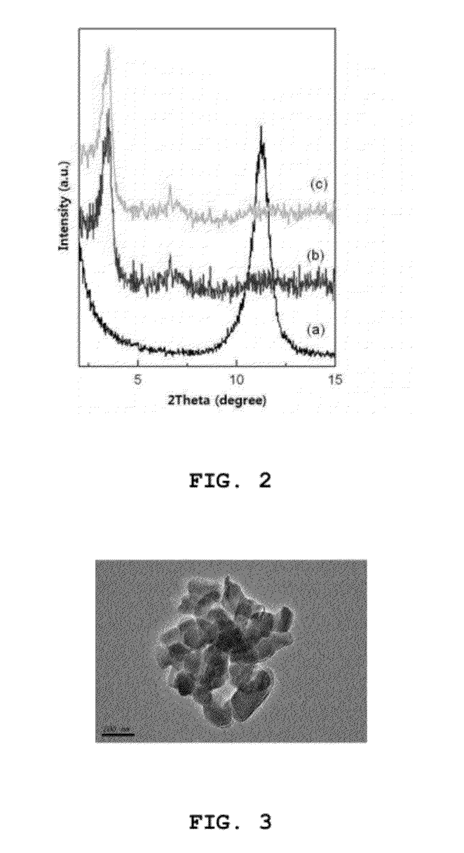

In order to examine the crystal structure of the nanohybrid prepared in Example 1, the nanohybrid was analyzed by X-ray diffraction (Rigaku, D/Max 2200). As a result, as can be seen in FIG. 2, the interlayer distance of the layered inorganic hydroxide was about 7.9 Å, indicating that the hydroxide is a typical layered structure having a nitrate anion intercalated therein. Also, the interlayer distance of the siRNA-layered inorganic hydroxide was about 25 Å, indicating that the nitrate anion was ion-exchanged with the siRNA and that the siRNA was parallel with the hydroxide layers and intercalated between the hydroxide layers while it was expanded to about 20 Å. This suggests that an siRNA-layered inorganic hydroxide nanohybrid having a two-dimensional structure was obtained. In addition, the target-specific, siRNA/layered inorganic hydroxide maintained the interlayer distance even after the multifunctional ligand was bonded thereto, suggesting that a target-specific, siRNA/layered inorganic hydroxide having an siRNA intercalated between the layers was prepared.

In order to examine the shape and particle size of the target-specific, siRNA/layered inorganic hydroxide nanohybrid prepared in Example 1, the hybrid was analyzed with a transmission electron microscope (JEOL JEM-2100F). As a result, as can be seen in FIG. 3, the target-specific, siRNA-layered inorganic hydroxide nanohybrid prepared in Example 1 had a mean particle size of 100±20 nm and was hexagonal in shape.

### Test Example 1

### Analysis of Stability of Target-Specific, siRNA/Layered Inorganic Hydroxide Nanohybrid in Serum

The stability of the target-specific siRNA-layered inorganic hydroxide nanohybrid (prepared in Example 1) in serum was examined.

Specifically, 10 μg (on an siRNA basis) of the nanohybrid was added to 90 μl of a stability test solution (containing 10% rat serum; Invitrogen) and allowed to stand at 37° C. 0, 0.5, 1, 2, 4, 6, 8, 10, 12 and 24 hours after addition of the nanohybrid, 12 μl of a sample was taken from the solution and immediately freeze-dried at −70° C. 2.5 μl of each of the samples was gel-electrophoresed (1% agarose gel) in Tris-acetate (TAE) buffer in order to determine whether the siRNA was stably maintained in the serum. As a control, pure siRNA was used.

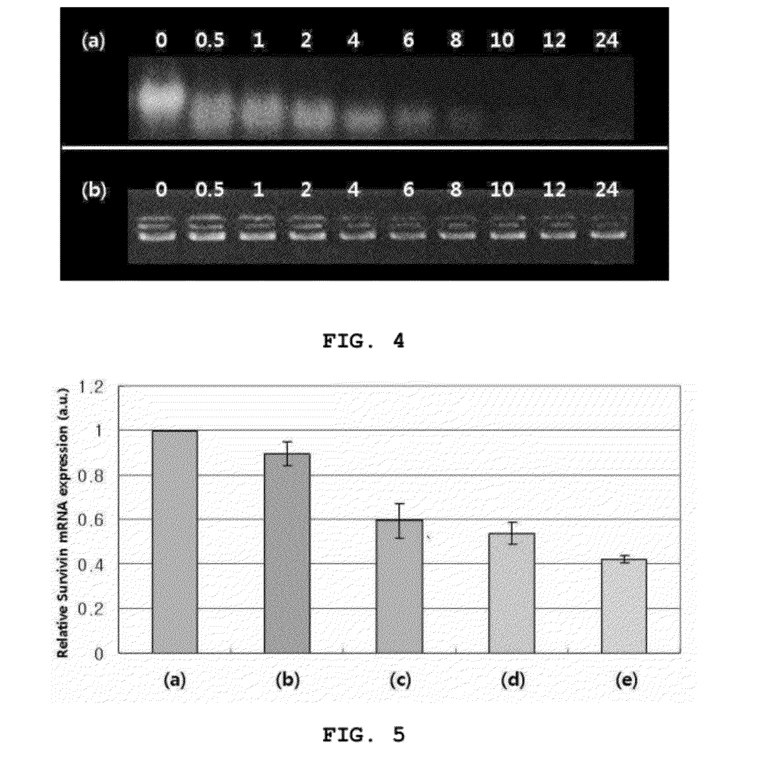

As a result, as shown in FIG. 4, the pure siRNA was substantially completely degraded when incubated in the serum-containing test solution for 8 hours (see FIG. 4(a)). On the other hand, in the case of the target-specific, siRNA/layered inorganic hydroxide nanohybrid, the nuclease-mediated degradation of the siRNA was not detected after 24 hours under the same conditions (see FIG. 4(b)). It is believed that this continued stability of the siRNA is obtained because the anionic phosphate group of the siRNA intercalated between the layers of the layered inorganic hydroxide is bonded to the cationic layer charge of the layered inorganic hydroxide by strong electrostatic interaction, and prevents nuclease from approaching the center of the inside of the nanohybrid.

### Example 2

### Examination of In Vitro Effects of Target-Specific, siRNA/Layered Inorganic Hydroxide Nanohybrid

2-1: Culture of Tumor Cell Line and Inhibition of Survivin Expression in the Tumor Cell Line

A human oral cancer cell line (KB, the Korean Cell Line Bank) overexpresses the folate receptor. In order to induce the maximum expression of the folate receptor, the cell line was cultured in a folate-free medium for 2 weeks or more under the conditions of 37° C. and CO₂.

The KB cells were dispensed in RPMI 1640 medium (Welgene, KR) at a density of 1×10⁵ cells/2 ml and cultured in a CO₂ incubator at 37° C. Then, the cells were treated with 100 nM (on an siRNA basis) of each of the siRNA/layered inorganic hydroxide nanohybrid and target-specific, siRNA/layered inorganic hydroxide nanohybrid prepared in Example 1. The treated cells were cultured in a CO₂ incubator at 37° C. After 6 hours, the cells were washed twice with RPMI 1640 medium, and the culture medium was replaced with fresh RPMI 1640 medium, after which the cells were additionally cultured in a CO₂ incubator at 37° C. for 24 hours in order to inhibit the expression of survivin.

As a comparative group, cells not treated with anything were used, and as a control group, cells treated with the NO₃/layered inorganic hydroxide. As a test group, cells treated with the siRNA/layered inorganic hydroxide nanohybrid were used.

In addition, in order to examine the tumor-specific endocrytosis with the target-specific ligand folic acid, cells which were cultured in folic acid (1 mg/ml)-containing medium for 24 hours and then treated with the target-specific, siRNA/layered inorganic hydroxide nanohybrid were used as a control group.

The target-specific, siRNA/layered inorganic hydroxide nanohybrid according to the present invention underwent receptor-mediated endocytosis in the cells in which the tumor marker folate receptor was over-expressed, thereby exhibiting tumor therapeutic effects. In addition, both the siRNA/layered inorganic hydroxide nanohybrid and the target-directed, siRNA/layered inorganic hydroxide nanohybrid were internalized by clathrin-mediated endocytosis in the cells in which the tumor marker was not expressed. Further, when the nanohybrid having the target-specific multifunctional ligand attached thereto was used, a large amount of the nanohybrid entered the cells in a tumor-selective manner. This suggests that the target-specific layered inorganic hydroxide can serve as a tumor cell-selective siRNA transfer mediator.

2-2: Quantitative Analysis of Survivin mRNA by RT-PCR

In order to confirm whether the inhibition of tumor cells is actually attributable to a decrease in the intracellular level of survivin mRNA, the concentration of survivin mRNA in total RNA was quantified using an RNA extraction kit (RNeasy mini kit, Qiagen, Germany) by RT-PCR (Real-time PCR) analysis in the following manner.

1 μg of total RNA from each sample was mixed with 1 μl of oligo-dT18 (500 ng/μl) and 2 μl of dNTP (each 2.5 mM) and allowed to react at 70° C. for 10 minutes. Then, the reaction material was cooled on ice for 5 minutes and mixed with 0.5 μl of reverse superscript (200 U/μl) (Invitrogen), 2 μl of 10× reaction buffer, 0.5 μl of RNase inhibitor and a suitable amount of water to make a total volume of 20 μl. Then, the mixture was allowed to react at 42° C. for 15 minutes, at 95° C. for 5 minutes and at 4° C. for 5 minutes, thereby obtaining cDNA. Then, 1 μl of the cDNA was mixed with 10 μl of 2×SYBR green master mix of a RT-PCR system (Applied Biosystems Prism 7900 Sequence Detection System, Applied Biosystems, USA), and 0.4 μl (10 pM) of each of forward and reverse primers specific for survivin and GAPDH, and then the mixture was subjected to RT-PCR in the following conditions: 40 cycles of 2 min at 50° C., 10 min at 95° C., 30 sec at 95° C., 30 sec at 60° C., and 30 sec at 72° C. The sequences of the forward and reverse primers used in the RT-PCR are as follows:

Survivin-specific forward primer:
(SEQ ID NO: 10)
5′-CCTTCACATCTGTCACGTTCTCC-3′
Survivin-specific reverse primer:
(SEQ ID NO: 11)
5′-ATCATCTTACGCCAGACTTCAGC-3′
GAPDH-specific forward primer:
(SEQ ID NO: 12)
5′-GGTGAAGGTCGGAGTCAACG-3′
GAPDH-specific reverse primer:
(SEQ ID NO: 13)
5′-ACCATGTAGTTGAGGTCAATGAAGG-3′

After completion of the PCR, the amount of the survivin PCR product and the amount of the GAPDH PCR product were measured using a cDNA standard curve, and the measured value of survivin was divided by the measured value of GAPDH, thereby determining the relative expression level of survivin. Based on the resulting value, a decrease in the expression of survivin mRNA was determined.

The results of the test are shown in FIG. 5. As can be seen therein, treatment of the KB cells with the siRNA/layered inorganic hydroxide nanohybrid resulted in a 40% decrease in survivin expression, and treatment of the KB cells with the target-directed, siRNA/layered inorganic hydroxide nanobrid resulted in a 60% decrease in survivin expression.

This suggests that the siRNA/layered inorganic hydroxide nanohybrid and target-specific, siRNA/layered inorganic hydroxide nanohybrid of the present invention can not only reduce the expression level of survivin mRNA, but also directly induce the inhibition of proliferation of tumor cells as a result of the decrease in the expression of survivin.

As described above, the target-specific, siRNA-layered inorganic hydroxide nanohybrid of the present invention increased the in vivo stability of siRNA, and the target-specific multifunctional ligand that can bind specifically to a tumor marker increased the efficiency of tumor-specific transfer of siRNA such that siRNA shows tumor therapeutic activity even at a relatively low dose. Thus, the nanohybrid of the present invention can be used as a composition which increases the efficiency and accuracy of treatment of various tumor diseases. In addition, it was confirmed that the nanohybrid of the present invention is a new type of siRNA delivery system which is very useful for basic bioengineering research and in the medical industry.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described above in detail, the target-specific, siRNA/layered inorganic hydroxide nanohybrid according to the present invention increases the in vivo stability of siRNA, and the target-specific multifunctional ligand that can bind specifically to a tumor marker increases the efficiency of tumor-specific transfer of siRNA such that siRNA can show tumor therapeutic activity at a relatively low dose. Thus, the nanohybrid of the present invention can be used as a composition which increases the efficiency and accuracy of treatment of various tumor diseases. In addition, the nanohybrid of the present invention is a new type of siRNA delivery system which is very useful for basic bioengineering research and in the medical industry.

1. A target-specific, siRNA/layered inorganic hydroxide nanohybrid represented by the following formula 1:
[M(II)₁₋ₓM(III)ₓ(OH)₂]^{X+}[S][T] [Formula 1]
wherein M(II) represents a divalent metal cation, M(III) represents a trivalent metal cation, x is a number ranging from 0.1 to 0.5, S is siRNA, and [T] is a tumor-targeted multifunctional ligand.

2. The target-specific, siRNA/layered inorganic hydroxide nanohybrid of claim 1, wherein the siRNA is a survivin-derived gene.

3. The target-specific, siRNA/layered inorganic hydroxide nanohybrid of claim 1, wherein the siRNA is any one nucleotide sequence selected from the group consisting of SEQ ID NOs: 1 to 9.

4. The target-specific, siRNA/layered inorganic hydroxide nanohybrid of claim 1, wherein the divalent metal cation is selected from the group consisting of Mg²⁺, Ca²⁺, Co²⁺, CU²⁺, Ni²⁺ and Zn²⁺, and the trivalent metal cation is selected from the group consisting of Al³⁺, Cr³⁺, Fe³⁺, Ga³⁺, In³⁺, V³⁺ and T³⁺.

5. The target-specific, siRNA/layered inorganic hydroxide nanohybrid of claim 1, wherein the tumor-targeted multifunctional ligand can bind specifically to any one selected from the group consisting of antigen, antibody, RNA, DNA, hapten, avidin, streptavidin, neutravidin, protein A, protein G, lectin, selectin, a radioisotope-labeled biomaterial, and tumor receptor.

6. The target-specific, siRNA/layered inorganic hydroxide nanohybrid of claim 5, wherein the tumor receptor is selected from the group consisting of ligands, antigens, receptors, and nucleic acids that encode them.

7. The target-specific, siRNA/layered inorganic hydroxide nanohybrid of claim 6, wherein the tumor receptor is selected from the group consisting of synaptotagmin I C2, annexin V, integrin, VEGF (Vascular Endothelial Growth Factor), angiopoietin 1, angiopoietin 2, somatostatin, vasointestinal peptide, carcinoembryonic antigen, HER2/neu antigen, prostate-specific membrane antigen, and folic acid receptor.

8. The target-specific, siRNA/layered inorganic hydroxide nanohybrid of claim 7, wherein the tumor-targeted multifunctional ligand that can bind specifically to the tumor receptor is one or more selected from the group consisting of phosphatidylserine, VEGFR, integrin receptor, Tie2 receptor, somatostatin receptor, vasointestinal peptide receptor, Herceptin, Rituxan, and folic acid receptor.

9. A pharmaceutical composition for tumor treatment, which contains the target-specific, siRNA/layered inorganic hydroxide nanohybrid of claim 1 together with a pharmaceutically acceptable carrier.

10. The pharmaceutical composition of claim 9, wherein the pharmaceutically acceptable carrier is one or more selected from the group consisting of ion exchange resin, alumina, aluminum stearate, lecithin, serum proteins, buffering agents, water, salts, electrolytes, colloidal silica, magnesium trisilicate, polyvinylpyrrolidone, cellulose-based substrates, polyethylene glycol, sodium carboxymethylcellulose, polyarylate, waxes, polyethylene glycol, and wool fat.

11. The pharmaceutical composition of claim 9, further containing additives selected from the group consisting of excipients, disintegrants, binders, lubricants, suspending agents, surfactants, sweeteners, preservatives, flavoring agents, thickeners, pH-adjusting agents, wetting agents, and mixtures thereof.

12. The pharmaceutical composition of claim 9, wherein the formulation is be selected from the group consisting of tablets, capsules, liquids, injectable solutions, ointments, and syrups.

13. The pharmaceutical composition of claim 9, wherein the formulation is an injectable solution, which is in the form of a liquid, a suspension or an emulsion.

14. The pharmaceutical composition of claim 9, which is formulated to be administered intravenously, intramuscularly, intra-arterially, intramedularry, intrathecally, intraventricularly, transdermally, subcutaneously, intraperitoneally, enterally, sublingually, or topically.

15. The pharmaceutical composition of claim 9, which is formulated in the form of a unit-dosage or multi-dosage container.

16. The pharmaceutical composition of claim 9, which contains 0.05 to 0.1 μg of siRNA per kg weight of a subject to be treated.

17. The pharmaceutical composition of claim 9, wherein the tumor is oral cancer or lung cancer.

18. A method for preparing a target-specific, siRNA/layered inorganic hydroxide nanohybrid, the method comprising the steps of: (a) adding an aqueous solution of a base dropwise to an aqueous solution containing a divalent metal salt and a trivalent metal salt to prepare a precipitated layered inorganic hydroxide; (b) mixing an siRNA-containing solution with a dispersion of the layered inorganic hydroxide prepared in step (a), and stirring the mixture, thereby preparing an siRNA/layered inorganic hydroxide nanohybrid; and (c) bonding a tumor marker-specific multifunctional ligand to the nanohybrid, thereby preparing a target-specific, siRNA/layered inorganic hydroxide nanohybrid.

19. The method of claim 18, wherein the layered inorganic hydroxide in step (a) is represented by the following formula 2:
[M(II)₁₋ₓM(III)ₓ(OH)₂]^{X+}[Aⁿ⁻]_{X/n}.yH₂O [Formula 2]
wherein M(II) represents a divalent metal cation, M(III) represents a trivalent metal cation, A is an anionic chemical species, n is the charge number of the anion, x is a number range from 0.1 to 0.5, and y is a positive number greater than 0.

20. The method of claim 19, wherein the divalent metal cation is selected from the group consisting of Mg²⁺, Ca²⁺, Co²⁺, Cu²⁺, Ni²⁺ and Zn²⁺, the trivalent metal cation is selected from the group consisting of Al³⁺, Cr³⁺, Fe³⁺, Ga³⁺, In³⁺, V³⁺ and Ti³⁺, and the anion is selected from the group consisting of CO₃²⁻, NO³⁻, Cl⁻, OH⁻, O²⁻ and SO₄²⁻.

21. A method for preparing a pharmaceutical composition for tumor treatment containing the target-specific, siRNA/layered inorganic hydroxide nanohybrid of claim 9, the method comprising step of: (a) adding an aqueous solution of a base dropwise to an aqueous solution containing a divalent metal salt and a trivalent metal salt to prepare a precipitated layered inorganic hydroxide; (b) mixing an siRNA-containing solution with a dispersion of the layered inorganic hydroxide prepared in step (a), and stirring the mixture, thereby preparing an siRNA/layered inorganic hydroxide nanohybrid; (c) bonding a tumor marker-specific multifunctional ligand to the nanohybrid, thereby preparing a target-specific, siRNA/layered inorganic hydroxide nanohybrid; (d) formulating the nanohybrid with a pharmaceutically acceptable carrier.
